(19) United States
(12) Patent Application Publication
(10) Pub. No.: US 2026/0279506 A1
(43) Pub. Date: Sep. 17, 2026

(54) METHOD AND SYSTEM FOR GENERATING FOOD FORMULATIONS

(71) Applicant: Sustento Inc., Toronto (CA)

(72) Inventors: Sofia Bonilla, Mississauga (CA); Alexandra Camara, Paris (CA)

(73) Assignee: Sustento Inc., Toronto (CA)

(21) Appl. No.: 19/463,628

(22) Filed: Jan. 29, 2026

(30) Foreign Application Priority Data
Mar. 6, 2025 (CA) 3266903

(51) Int. Cl. G16C 20/20; G16C 20/70; G16C 20/90

(52) U.S. Cl. CPC G16C 20/20; G16C 20/70; G16C 20/90

(57) ABSTRACT

The present disclosure provides a system, a method and a non-transitory computer-readable storage medium for generating food formulations is disclosed. The method includes receiving one or more input parameters from a user. The method includes retrieving a plurality of pre-defined regulatory guidelines from one or more sources. The method includes storing a set of data. The method includes analyzing, using a machine learning-based model, the one or more input parameters, the plurality of pre-defined pet nutrition-based regulatory guidelines, and the set of data. Further, the method includes determining, using the ML model, nutrient retention time and nutrient degradation time for a selected food processing type based on the analysis of the one or more input parameters, the plurality of pre-defined pet nutrition-based regulatory guidelines, and the set of data. The method includes generating at least one food formulation of a plurality of food formulations.

### CROSS-REFERENCE TO RELATED APPLICATIONS

The present application claims the benefit of Canadian Patent Application No. 3266903, filed on Mar. 6, 2025, all of which are hereby incorporated by reference in their entirety for all purposes.

### TECHNICAL FIELD

The present disclosure relates to the field of machine learning, and in particular, relates to method and system for generating food formulations using machine learning-based models.

### BACKGROUND

The food industry has seen significant advancements in recent years, driven by growing consumer demand for high-quality, nutritionally optimized, and environmentally sustainable products. Modern pet owners increasingly seek solutions that address specific dietary and health needs while ensuring transparency, functionality, and compliance with established standards. This shift has prompted the pet food industry to focus on developing pet food formulations that balance nutrition, palatability, sustainability, and cost-effectiveness. However, the existing animal and pet food formulation techniques often rely on manual methods or limited datasets, which can lead to inefficiencies, higher costs, and suboptimal product outcomes. The existing formulation techniques fail to address the complex interdependencies among nutrition, processing impacts, regulatory compliance, sustainability, and cost.

To overcome the above-mentioned limitations, there is a need for an advanced solution for generating animal and pet food formulations using machine learning model.

### SUMMARY

In an aspect, a system for generating food formulations is disclosed. The system includes an input module configured to receive one or more input parameters from a user. The system includes a regulatory data integration module configured to retrieve a plurality of pre-defined regulatory guidelines from one or more sources. The system includes a database configured to store a set of data. The set of data comprises at least a plurality of nutritional profiles, environmental impact metrics, cost-related data, palatability metrics, and historical nutrient retention coefficients. The system also includes a processing unit configured to analyze, using a machine learning-based model, the one or more input parameters, the plurality of pre-defined regulatory guidelines, and the set of data. The processing unit is further configured to determine, using the machine learning-based model, nutrient retention time and nutrient degradation time for a selected food processing type of a set of food processing types based on the analysis of the one or more input parameters, the plurality of pre-defined nutrition-based regulatory guidelines, and the set of data. The processing unit is further configured to generate, using the ML model, at least one food formulation of a plurality of food formulations based on the determined nutrient retention time and the nutrient degradation time.

In an embodiment of the present disclosure, the plurality of food formulations includes one of: pet food formulations and animal feed formulations.

In an embodiment of the present disclosure, the one or more input parameters includes animal and pet category, animal and pet food habits, intended nutritional goals, geographical location, ingredient preferences, ingredient exclusions, and the set of food processing types.

In an embodiment of the present disclosure, the set of data is extracted from the one or more sources. The one or more sources includes at least one of: regulatory databases, ingredient databases, cost datasets, environmental impact datasets, and animal and pet behaviour studies.

In an embodiment of the present disclosure, the processing unit is configured to rank, using the machine learning-based model, the plurality of food formulations based on a set of user preferences. The set of user preferences is determined based on the one or more input parameters received from the user.

In an embodiment of the present disclosure, the processing unit is configured to generate, using the machine learning-based model, a detailed report for each of the plurality of food formulations depicting at least one of: nutrient profile, ingredients list, cost and sustainability metrics.

In an embodiment of the present disclosure, the detailed report includes comparison of the generated plurality of food formulations against the plurality of pre-defined regulatory guidelines. In addition, the detailed report includes a list of one or more functional claims and one or more packaging-ready statements derived from one or more nutritional and functional attributes of the plurality of food formulations. Further, the detailed report includes sustainability metrics and potential certifications comprising at least carbon footprint, water efficiency, and environmental metrics.

In an embodiment of the present disclosure, the machine learning-based model is trained using pre-historic data and real-time data associated with the one or more input parameters. The pre-historic data is stored in the database and the real time data is received from at least one of: the one or more sources and the user.

In an embodiment of the present disclosure, the processing unit is further configured to generate, using the machine learning-based model, one or more recommendations for reformulating one or more existing food formulations, based on at least a scientific research, regulatory updates, change in existing user preferences, and market pricing trends.

In an embodiment of the present disclosure, the database is periodically updated based on at least a scientific research, regulatory updates, change in existing user preferences, and market pricing trends.

In another aspect, a method for generating food formulations is disclosed. The method includes receiving one or more input parameters from a user. The method includes retrieving a plurality of pre-defined regulatory guidelines from one or more sources. The method includes storing a set of data. The set of data comprises at least a plurality of nutritional profiles, environmental impact metrics, cost-related data, palatability metrics, and historical nutrient retention coefficients. In addition, the method includes analysing, using a machine learning-based model, the one or more input parameters, the plurality of pre-defined nutrition-based regulatory guidelines, and the set of data. Further, the method includes determining, using the machine learning-based model, nutrient retention time and nutrient degradation time for a selected food processing type of a set of food processing types based on the analysis of the one or more input parameters, the plurality of pre-defined pet nutrition-based regulatory guidelines, and the set of data. The method includes generating, using the machine learning-based model, at least one food formulation of a plurality of food formulations based on the determined nutrient retention time and the nutrient degradation time.

In yet another aspect, a non-transitory computer-readable medium storing one or more instructions for generating the food formulations is disclosed. The one or more instructions which, when executed by a processor, causes the processor to receive the one or more input parameters from the user. The one or more instructions which, when executed, causes the processor to retrieve the plurality of pre-defined regulatory guidelines from the one or more sources. In addition, the one or more instructions which, when executed, causes the processor to store a set of data, wherein the set of data comprises at least a plurality of nutritional profiles, environmental impact metrics, cost-related data, and historical nutrient retention coefficients. Further, the one or more instructions which, when executed, causes the processor to analyse, using the machine learning-based model, the one or more input parameters, the plurality of pre-defined nutrition-based regulatory guidelines, and the set of data. Also, the one or more instructions which, when executed, causes the processor to determine, using the machine learning-based model, the nutrient retention time and nutrient degradation time for a selected food processing type of a set of food processing types. The nutrient retention time and the nutrient degradation time is determined based on the analysis of the one or more input parameters, the plurality of pre-defined nutrition-based regulatory guidelines, and the set of data. The one or more instructions which, when executed, causes the processor to generate, using the machine learning-based model, at least one food formulation of the plurality of food formulations based on the determined nutrient retention time and the nutrient degradation time.

### BRIEF DESCRIPTION OF THE FIGURES

For a better understanding of the various described embodiments, reference should be made to the Detailed Description below, in conjunction with the following drawings in which like reference numerals refer to corresponding parts throughout the figures.

FIG. 1 illustrates an interactive computing environment for machine learning-based generation of food formulations, in accordance with various embodiments of the present disclosure;

FIG. 2 illustrates an exemplary overview of a system for generating the food formulations, in accordance with various embodiments of the present disclosure;

FIG. 3 illustrates a flow chart of a method for generating the food formulations, in accordance with various embodiments of the present disclosure; and

FIG. 4 illustrates a block diagram of a computing device, in accordance with various embodiments of the present disclosure.

In accordance with common practice the various features illustrated in the drawings may not be drawn to scale. Accordingly, the dimensions of the various features may be arbitrarily expanded or reduced for clarity. In addition, some of the drawings may not depict all of the components of a given system, method, or device. Finally, like reference numerals may be used to denote features throughout the specification and figures.

### DETAILED DESCRIPTION

In the following description of the disclosure and embodiments, reference is made to the accompanying drawings in which it is shown by way of illustration of specific embodiments that can be practiced. It is to be understood that other embodiments and examples can be practiced, and changes can be made without departing from the scope of the disclosure.

Although the following description uses the terms “first,” “second,” etc. to describe various elements, these elements should not be limited by the terms. These terms are only used to distinguish one element from another. For example, a first input could be termed a second input, and, similarly, a second input could be termed a first input, without departing from the scope of the various described examples. The first input and the second input can both be outputs and, in some cases, can be separate and different inputs.

The terminology used in the description of the various described examples herein is for the purpose of describing specific examples only and is not intended to be limiting. As used in the description of the various described examples and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

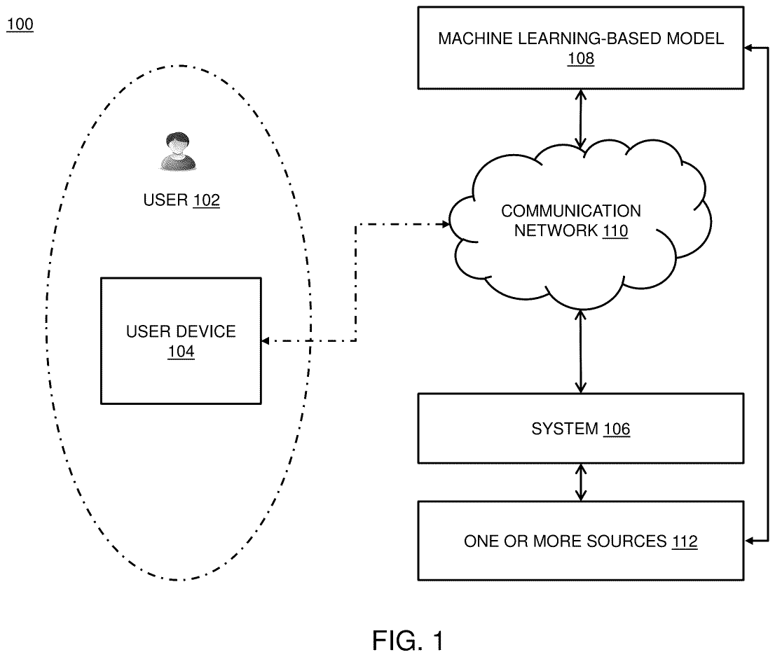

FIG. 1 illustrates an interactive computing environment 100 for machine learning-based generation of food formulations, in accordance with various embodiments of the present disclosure. The interactive computing environment 100 includes a user 102, a user device 104 associated with the user 102, a system 106, a machine learning-based model 108 and one or more data sources 112 associated with the system 106, connected to, and in communication with (and/or with access to) a communication network (e.g., a communication network 110). The components of the interactive computing environment 100 are associated with each other and function properly to perform the machine learning-based generation of the food formulations.

The user 102 represents an entity engaging with the system 106 to generate a plurality of food formulations. The plurality of food formulations includes but may not be limited to pet food formulations and animal feed formulations. In an example, the user 102 may be a pet food manufacturer seeking customized pet food formulations. In another example, the user 102 may be a veterinarian or pet nutritionist designing specialized pet food formulations for pets with medical conditions. In yet another example, the user 102 may be an individual pet owner creating pet food formulations tailored to a pet's specific needs. In yet another embodiment, the user may be a caretaker in an animal care centre. The user 102 provides one or more input parameters to the system 106.

The one or more input parameters includes but may not limited to animal and pet category, animal and pet food habits, intended nutritional goals, geographical location of the pet, ingredient preferences, ingredient exclusions, and the set of food processing types. The animal and pet category refers to classification of animals and pets based on their species, age, size, breed, or specific life stage. The animal and pet category helps in determining appropriate nutritional requirements for generation of the plurality of food formulations. In an example, the animal and pet category may include species-based classification such as dogs, cats, rabbits, birds and the like. In another example, the animal and pet category may include age-based classification such as puppies/kittens (growth stage), adult pets (maintenance stage), senior pets (aging stage). In yet another example, the animal and pet category may include size-based classification such as small breeds (e.g. Chihuahuas, Persian cats, etc.). In yet another example, the animal and pet category includes specialized category based on health conditions such as overweight or obese animals or pets, animals and pets with food allergies, animals and pets with kidney disease, etc. Further, animal and pet food habits refer to feeding behaviors, dietary preferences, and consumption patterns of animals and pets.

Also, the geographical location of the animal and pet is utilized to determine its dietary requirements, and ingredient availability for generation of the food formulation. The geographical location of the animal and pet influences selection of ingredients, nutrient composition, and feeding strategies to ensure the pet's health and well-being in different environmental conditions. The user 102 provides this as an input parameter to the system 106, allowing the generation of food formulations tailored to location-specific needs. For example, pets in colder climates (e.g. Canada, Russia, etc.) require high-calorie diets to maintain body heat. In addition, animals in hot and humid regions (e.g. Deserts, etc.) need hydration focused diets with moisture-rich foods such as wet foods, or hydrating supplements.

Generally, food processing types (in context of animal and pet food) refers to methods used to manufacture food, each of which affects final product's nutritional composition texture, palatability, and shelf stability. In an embodiment of the present disclosure, the set of food processing types may be dry extrusion, baked food, wet canned food, dehydrated food, and the like. For dry extrusion, high-temperature and high-pressure cooking of ingredients are required, followed by shaping and drying. For baked food, ingredients are mixed into a dough, baked at lower temperatures than extrusion, and then cooled. For wet canned foods, ingredients are mixed, cooked, sealed in cans, and sterilized through retort cooking. In addition, for dehydrated food slow removal of moisture is done at low temperatures while preserving nutrients. Each food processing type of the set of food processing types influence the food formulations, necessitating specific adjustments in ingredient selection, nutrient fortification, and compliance checks to maintain nutritional integrity.

The food formulations refer to a structured combination of ingredients tailored to meet specific nutritional, cost, and regulatory requirements. The formulation process balances one or more competing objectives. The one or more competing objectives include at least nutritional adequacy, cost efficiency, palatability and sustainability. The nutritional adequacy ensures essential vitamins, minerals, proteins, and fats that meet pet dietary needs. The cost efficiency corresponds to optimizing ingredient selection to minimize production costs while maintaining quality. The palatability corresponds to enhancement of texture, aroma, and flavour to increase pet acceptance. The sustainability corresponds to evaluating the environmental footprint of ingredients (e.g., greenhouse gas emissions, water consumption, etc.).

The user 102 utilizes the user device 104, which serves as an interface for submitting food formulation requests and receiving the plurality of food formulations as output. In an example, the user device 104 includes a smartphone, tablet, personal computer, laptop, or an IoT-enabled food dispenser that automates feeding schedules based on optimized formulations. The user device 104 may include other communication devices equipped with input/output components such as keyboards, touchscreens, and microphones, enabling comprehensive data exchange between the user device 104 and the system 106.

In an embodiment, the user device 104 is a fixed communication device. In another embodiment, the user device 104 is a portable communication device. In some examples, the fixed communication device includes, but is not limited to, desktop computers, smart kiosks, or wall-mounted terminals. In some examples, the portable communication device includes, but is not limited to, smartphones, tablets, laptops, handheld gaming devices, or wearable devices such as smartwatches and augmented reality glasses.

The user device 104 runs on an operating system such as iOS, Android, or Windows, providing the necessary software components for data processing and system management. The operating system also facilitates communication between hardware and software components, ensuring smooth interaction between the user device 104 and the system 106.

In one implementation, the user device 104 may run on an operating system stored in a memory of the user device 104. In some examples, the operating system may include Darwin, RTXC, LINUX, UNIX, OS X, iOS, WINDOWS, an embedded operating system, and the like. In addition, the user device 104 may run on any version of the operating system. In general, the operating system includes various software components and/or drivers for controlling and managing general system tasks (e.g., memory management, storage device control, power management, etc.) and facilitates communication between various hardware and software components.

The system 106 serves as a central processing unit for executing food formulation requests. In particular, the system 106 is configured to generate the plurality of food formulations. The system 106 is implemented as a cloud-based infrastructure, a distributed server network, or a standalone on-premise computing cluster.

The primary functions of the system 106 include receiving the one or more input parameters from the user 102, retrieving a plurality of pre-defined regulatory guidelines from the one or more sources 112, storing a set of data extracted from the one or more sources 112, analysing the one or more input parameters, the plurality of pre-defined regulatory guidelines, and the set of data using the machine learning-based model 108, determining nutrient retention time and nutrient degradation time for a selected food processing type of the set of food processing types based on the analysis and generating at least one food formulation of the plurality of food formulations.

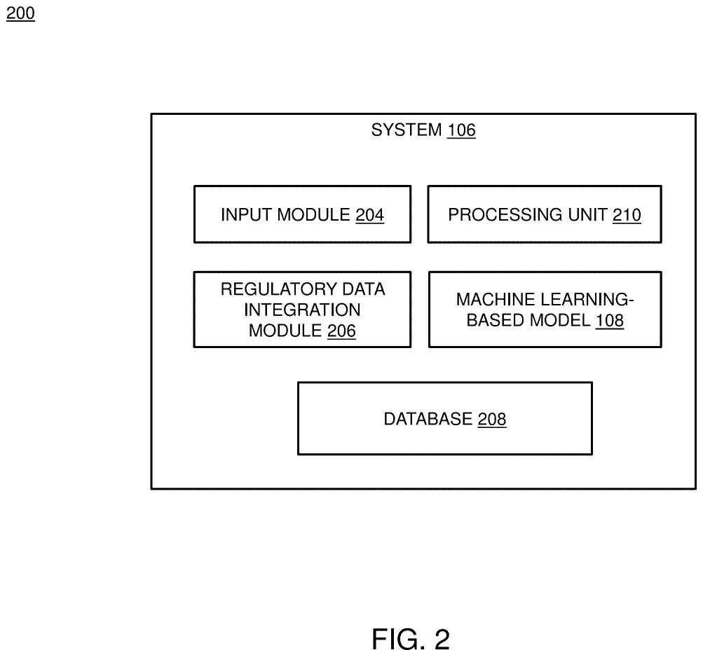

In addition, the system 106 is designed to generate the plurality of food formulations ensuring that the food formulations meet nutritional, cost-efficiency, and the plurality of pre-defined regulatory guidelines. Further, the system 106 is configured to rank the plurality of food formulations based on a set of user preferences. The set of user preferences is determined based on the one or more input parameters received from the user 102. In addition, the system 106 generates a detailed report for each of the plurality of food formulations depicting at least one of nutrient profile, ingredient list, cost and sustainability metrics (further explained below in description of FIG. 2).

The system 106 interacts with the one or more sources 112 to retrieve the plurality of pre-defined regulatory guidelines. The one of more sources 112 includes at least one of regulatory databases, ingredient databases, cost datasets, environmental impact datasets, and animal and pet behaviour studies. Further, the system 106 interacts with the one or more sources 112 to extract the set of data. The set of data includes at least a plurality of nutritional profiles, environmental impact metrics, cost related data, and palatability metrics, historical nutrient retention coefficients (further explained below in the description of FIG. 2).

Further, the system 106 interacts with the machine learning-based model 108. The machine-learning-based 108 (hereinafter, “ML model 108”) is trained using pre-historic data and real-time data associated with the one or more input parameters to facilitate generation of the plurality of food formulations (explained further below in the description of FIG. 2).

The ML model 108 may use several machine learning algorithms to process data and generate the plurality of food formulations. These algorithms help the system 106 to balance multiple objectives like cost efficiency, sustainability, and nutritional adequacy.

In an embodiment, the ML model 108 uses supervised learning (e.g., Regression Models, Neural Networks, etc.). The system 106 uses labelled data to learn relationships between input ingredients and output formulations. For instance, the historical food formulations (with known costs, nutritional values, and user ratings) are fed into a neural network, which learns how to predict the best ingredient combinations based on a set of input parameters.

In another embodiment, the ML model 108 may use unsupervised learning (e.g., Clustering, Dimensionality Reduction). The system 106 uses unsupervised learning algorithms to identify hidden patterns in data. For example, clustering algorithms might identify groups of ingredients with similar nutritional profiles or cost characteristics, enabling the system 106 to suggest new ingredient combinations that meet user needs without labelling.

In an embodiment, the system 106 uses a multi-objective optimization algorithm. The system 106 applies the multi-objective optimization algorithm for generation of the food formulations balancing competing goals, such as minimizing costs while maximizing nutritional value.

The system 106 interacts with various components of the interactive computing environment using a communication network 110. The communication network 110 acts as a central communication infrastructure of the interactive computing environment 100. The communication network 110 provides seamless connectivity between the user device 104, the system 106, the machine learning-based model 108 and the one or more data sources 112. The communication network 110 supports various communication protocols, including but not limited to TCP/IP, UDP, 2G, 3G, 4G, 5G, LTE, and other future protocols, enabling reliable data transfer between different components in the system 106. The communication network 110 facilitates real-time interaction, enabling generation of the plurality of food formulations while maintaining cost efficiency, sustainability, nutritional adequacy, and palatability.

The user device 104 communicates with the system 106 through the communication network 110 to provide the one or more input parameters to the system 106. The communication network 110 ensures that the one or more input parameters, along with the plurality of pre-defined regulatory guidelines is transferred securely to the system 106.

Architecture of the system 106 supports flexible configurations, allowing different types of systems or devices to perform the functions described above. In some implementations, the system 106 may be any of a general purpose device such as a computer or a special purpose device such as a client, a server, and the like. Any of these general or special purpose devices may include any suitable components such as a hardware processor (which can be a microprocessor, digital signal processor, a controller, etc.), memory, communication interfaces, display controllers, input devices, and the like. The system 106 may be deployed as a cloud-based system, an edge device, or an embedded device within the user device 104. The communication network 110 ensures that these devices work seamlessly together, providing a scalable and efficient solution for the optimization of the food formulations.

The system 106 herein may correspond to a dedicated server, a cloud-based computing system, or an edge device embedded within the user device 104. In some implementations, the system 106 may be implemented as one server or can be distributed as any suitable number of servers. For example, multiple servers can be implemented in various locations to increase reliability, function of the application, and/or the speed at which the system 106 may communicate with the user device 104.

The number and arrangement of systems, devices, and/or networks shown in FIG. 1 are provided as an example. There may be additional systems, devices, and/or networks; fewer systems, devices, and/or networks; different systems, devices, and/or networks, and/or differently arranged systems, devices, and/or networks than those shown in FIG. 1. Furthermore, two or more systems or devices shown in FIG. 1 may be implemented within a single system or device, or a single system or device shown in FIG. 1 may be implemented as multiple, distributed systems or devices. Additionally, or alternatively, a set of systems or a set of devices of the interactive computing environment 100 may perform one or more functions described as being performed by another set of systems or another set of devices of the interactive computing environment 100.

The system 106 is further explained in detail in FIG. 2.

FIG. 2 illustrates an exemplary overview 200 of the system 106 for generating the food formulations, in accordance with various embodiments of the present disclosure. The system 106 includes one or more components that interact with each other to facilitate generation of a plurality of food formulations. The one or more components includes but may not be limited to an input module 204, a regulatory data integration module 206, a database 208, a processing unit 210, and the machine learning-based model 108. The input module 204 is configured to receive the one or more input parameters from the user 102. The one or more input parameters includes animal and pet category, animal and pet food habits, intended nutritional goals, geographical location, ingredient preferences, ingredient exclusions, and the set of food processing types.

In an example, the user 102 accesses a web-based platform or mobile application on the user device 104 to upload the one or more input parameters. Further, the regulatory data integration module 206 is configured to retrieve the plurality of pre-defined regulatory guidelines from the one or more sources 112. The regulatory data integration module 206 interacts with the one or more sources 112 to retrieve the plurality of pre-defined regulatory guidelines. The plurality of pre-defined regulatory guidelines are retrieved to ensure compliance with pet food industry standards, such as the AAFCO (Association of American Feed Control Officials) guidelines. For example, “The formulation must adhere to the nutritional guidelines for adult cat food as defined by AAFCO.

In an embodiment, the regulatory data integration module 206 retrieves the plurality of pre-defined regulatory guidelines based on the one or more input parameters from the one or more data sources 112. The plurality of pre-defined regulatory guidelines corresponds to established rules, standards, and best practices relevant to the food formulations. The plurality of pre-defined regulatory guidelines ensure that the resulting food formulations comply with regulatory frameworks in different jurisdictions.

The regulatory frameworks are related to various aspects of animal and pet food, such as nutritional composition, ingredient sourcing, sustainability standards, health and safety regulations, and environmental impact. The plurality of pre-defined regulatory guidelines is utilized by the ML model 108 to identify relevant regulatory guidelines based on the one or more input parameters. For example, if the user 102 sets a sustainability threshold, the ML model 108 prioritizes regulatory guidelines that address environmental impacts such as carbon footprint or water usage limits.

Further, the set of data is extracted from the one or more sources 112. The set of data includes at least plurality of nutritional profiles, environmental impact metrics, cost-related data, palatability metrics, and historical nutrient retention coefficients. The set of data is stored in the database 208. The nutritional profiles refer to detailed information about nutritional composition of various ingredients required in the food formulation.

The nutritional profiles include data on macro and micronutrients such as proteins, carbohydrates, fats, vitamins, and minerals, as well as other relevant aspects such as caloric density, digestibility, and biological value. The database 208 stores the nutritional profiles for each ingredient, allowing the ML model 108 to ensure the generated food formulations meets the specific nutritional requirements for target species (e.g., dog, cat). In an example: for chicken, the nutritional profile may include data such as protein content (e.g., 31% protein), fat content (e.g., 4%), and other vitamins and minerals essential for a healthy pet diet. The database 208 stores this information for all potential ingredients. The information is further utilized by the processing unit 210 and the ML model 108 to generate the plurality of food formulations.

The environmental impact metrics refer to data on environmental sustainability of ingredients to be used in the food formulations. The environmental impact metrics include data such as carbon footprint, water usage, energy consumption, and land use associated with producing and transporting ingredients. The processing unit 210 in association with the ML model 108 may generate the food formulations to align with user-defined sustainability goals (e.g., minimizing carbon emissions) by storing these metrics. In an example: the carbon footprint of different protein sources (e.g., beef, chicken, fish) varies significantly. The system 106 may store these metrics in the database 208 and prioritize ingredients that have a lower carbon footprint if the user 102 has set a sustainability goal.

The cost-related data herein refers to information on market cost of each ingredient, which is essential for generating optimized food formulations to meet cost-efficiency goals of the user 102. The cost-related data includes a price per unit of each ingredient (e.g., per kilogram or per ton) and may also account for factors such as supply chain fluctuations or market trends that influence prices. In an example: if the cost of salmon rises due to seasonal factors or market demand, the system 106 stores updated cost data to reflect real-time pricing of ingredients. If a user prioritizes cost efficiency, the ML model 108 may generate the food formulation by selecting ingredients based on the lowest cost while maintaining nutritional adequacy.

The palatability metrics refer to data on acceptability and appeal of various ingredients to animals and pets. The palatability metrics include sensory attributes like taste, texture, and smell, which influence a pet's willingness to consume the food. The system 106 stores these metrics for each ingredient and uses them to ensure that the generated food formulation is palatable to animals and pets, encouraging consistent consumption and improving overall pet health. In an example, for a dog food formulation, the system 106 may store the palatability metrics for ingredients like chicken, lamb, or turkey, with specific data on how dogs react to these flavors or textures.

The historical nutrient retention coefficients refer to scientifically derived values that indicate how much of a nutrient remains in an ingredient after undergoing a specific food processing method. These coefficients are derived based on past experimental data and industry studies that measure nutrient degradation during food processing methods (e.g., extrusion, baking, canning). The system 106 may store the historical nutrient retention coefficients for the ML model 108 to generate the plurality of food formulations that meet regulatory guidelines (AAFCO, etc.) while maintaining nutritional integrity despite losses associated with the set of food processing types. The ML model 108 applies these coefficients to predict the final nutrient composition of food after food processing.

In an example, if an ingredient contains 100 mg of Vitamin C per kg but undergoes an extrusion process with a 50% retention coefficient, the ML model 108 may estimate that only 50 mg/kg remains in the final food formulation.

The one or more sources includes at least one of: regulatory databases, ingredient databases, cost datasets, environmental impact datasets, and animal and pet behaviour studies. The regulatory databases ensure compliance with industry standards established by organizations such as the Association of American Feed Control Officials (AAFCO), the U.S. Food and Drug Administration (FDA), and the European Pet Food Industry Federation (FEDIAF). The ingredient databases include detailed profiles of raw materials, including nutrient values, allergen risks, and digestibility factors. The cost datasets provide real-time ingredient pricing, allowing for cost-effective generation of the pet food formulations.

The environmental impact datasets assess sustainability metrics such as greenhouse gas emissions and water consumption associated with ingredient sourcing. The animal and pet behavior studies contribute insights on palatability and consumption trends, ensuring that the food formulations align with animal and pet feeding preferences. In an embodiment, the one or more sources 112 may include government and regulatory body websites, such as those of the FDA, European Food Safety Authority (EFSA), or other relevant authorities. In addition, the one or more sources 112 include industry standards organizations like AAFCO or ISO (International Organization for Standardization). Further, the one or more sources 112 may include private databases maintained by regulatory or industry groups, which compile up-to-date standards and guidelines. Furthermore, the one or more sources 112 may include academic sources or peer-reviewed publications that detail new regulatory considerations and scientific advancements in the food formulations.

Further, the set of data is stored in the database 208. In an embodiment, the database 208 stores the one or more input parameters received from the user 102. In addition, the database 208 stores the retrieved pre-defined regulatory guidelines. The database 208 stores the one or more input parameters, the pre-defined regulatory guidelines, and the set of data for the machine learning-based model 108 to generate at least the food formulation of the plurality of food formulations. The database 208 provides the one or more input parameters, the plurality of pre-defined regulatory guidelines, and the set of data to the processing unit 210 and the machine learning-based model 108. The processing unit 210 analyses the one or more input parameters, the plurality of pre-defined regulatory guidelines, and the set of data using the machine learning-based model 108. The machine learning-based model 108 (hereinafter, “ML model 108”) utilises one or more machine learning algorithms to facilitate analysis of the one or more input parameters, the plurality of pre-defined regulatory guidelines, and the set of data.

Further, the processing unit 210 is configured to determine nutrient retention time and nutrient degradation time for a selected food processing type of the set of food processing types, using the ML model 108. The processing unit 210 determines the nutrient retention time and the nutrient degradation time based on the analysis of the one or more input parameters, the plurality of pre-defined nutrition-based regulatory guidelines, and the set of data. Further, the processing unit 210 is configured to generate at least one food formulation of the plurality of food formulations using the ML model 108 based on the determined nutrient retention time and the nutrient degradation time.

The nutrient retention time is an estimated time during processing when a nutrient remains stable and bioavailable before degradation starts. The nutrient degradation time refers to a point of time at which a nutrient starts breaking down due to heat, pressure, moisture, or other processing factors. For determining the nutrient retention time and the nutrient degradation time, the processing unit 210 evaluates how different food processing types of the set of food processing types impact the nutrient composition of animal and pet food. The processing unit 210 evaluates this using the ML model 108 that considers the one or more input parameters, the plurality of pre-defined regulatory guidelines and ingredient and processing data such as nutrient retention coefficients, historical degradation trends, and the like.

In an example, a pet food manufacturer wants to create high-protein dry kibble for adult dogs. The ML model 108 selects extrusion as a food processing type and analyses the nutrient retention time and the nutrient degradation time. For kibble, the ML model 108 determines that the nutrient retention time is 120 seconds as the ingredients remain stable for 120 seconds at 100 degrees Celsius. Further, the ML model 108 determines that the nutrient degradation time begins after 150 seconds with a 5% nutrient loss. Based on this determined information, the ML model 108 generates the food formulation with no fortification.

In one implementation, the database 208 may include a non-transitory memory that stores software instructions, algorithms, and pre-trained models (machine-learning-based model 108) used by the system 106. In an embodiment, the non-transitory memory stores essential data, including the one or more input parameters, user preferences, ingredient databases, nutritional information, market trends, and other relevant datasets. The non-transitory memory allows the system 106 to retrieve historical data, making it possible to generate optimized food formulations.

The non-transitory memory may include one or more computer-readable storage mediums. The computer-readable storage mediums may be tangible and non-transitory. For instance, the non-transitory memory may include high-speed random-access memory and may also include non-volatile memory, such as one or more magnetic disk storage devices, flash memory devices, or other non-volatile solid-state memory devices.

In some examples, a non-transitory computer-readable storage medium of the non-transitory memory can be used to store instructions (e.g., for performing aspects of process 300, described below) for use by or in connection with an instruction execution system, apparatus, or device, such as a computer-based system, processor-containing system, or other system that can fetch the instructions from the instruction execution system, apparatus, or device and execute the instructions. In other examples, the instructions (e.g., for performing aspects of process 300, described below) can be stored on a non-transitory computer-readable storage medium (not shown) of the system 106. In the context of this document, a “non-transitory computer-readable storage medium” can be any medium that can contain or store the program for use by or in connection with the instruction execution system, apparatus, or device.

The non-transitory memory stores training data used for training the ML model 108, ensuring the system 106 can continuously learn and improve based on past inputs and feedback received from the user 102. The ML model 108 is trained using the training data that includes pre-historic data and real-time data associated with the one or more input parameters. The pre-historic data is stored in the database 208 (or the non-transitory memory of the database 208) and the real time data is received from at least one of: the one or more sources 112 and the user 102. The database 208 is periodically updated based on at least a scientific research, regulatory updates, change in existing user preferences, and market pricing trends to update the training data for the ML model 108.

The processing unit 210 utilizes the ML model 108 to rank the plurality of food formulations based on a set of user preferences. The set of user preferences is determined based on the one or more input parameters received from the user. The set of user preferences may include factors such as animal and pet category, dietary needs, ingredient preferences, budget constraints, sustainability concerns, and specific health goals.

By leveraging the ML model 108, the processing unit 210 ensures that the most suitable food formulation is ranked highest, offering a customized and data-driven approach to pet nutrition. For example, a pet owner with a senior Golden Retriever suffering from joint issues may prefer a formulation that includes glucosamine and turmeric while avoiding artificial additives. These input parameters define the set of user preferences, which the ML model 108 processes to rank various food formulations. The ML model 108 evaluates multiple food formulations and assigns ranking scores based on how well the formulations align with the user preferences. For example, the pet food formulations that meet pet's nutritional needs, fit within the budget, and match ingredient preferences receive higher rankings, whereas those that include undesired ingredients or exceed budget limits rank lower. In another example, if a formulation contains sustainably sourced fish oil, turmeric for joint health, and meets organic ingredient requirements, it will be ranked higher compared to a formulation that relies on meat by-products and artificial additives.

In an example, the ML model 108 processes the one or more input parameters and generates five distinct food formulations, ranking them based on the set of user preferences. Suppose a pet owner is looking for a balanced, grain-free diet for their adult “Labrador Retriever” dog with a focus on joint health, budget friendly, and sustainability. Based on these inputs, the ML model 108 evaluates multiple formulations and ranks them according to nutrient profile, ingredient suitability, cost, and sustainability metrics.

The top-ranked formulation, “Grain-Free Joint Support Formula”, is specifically designed for large breed dogs with joint health concerns. This formulation features high-quality protein from deboned chicken, omega-3 fatty acids from salmon oil, and glucosamine for joint support. This formulation is grain-free, and can be made using sweet potatoes and lentils as carbohydrate sources. This formulation is ranked first because it aligns perfectly with the pet owner's preferences, offering balanced nutrition while remaining affordable and sustainably sourced.

Further, second-ranked formulation, “High-Protein Mobility Formula”, contains a blend of chicken and fish meal, enhanced with turmeric extract and green-lipped mussel for anti-inflammatory benefits. While it provides excellent joint support and high protein content, it is slightly more expensive than the top-ranked option, leading to its second-place ranking.

Third ranked formulation, “Holistic Wellness Recipe”, is a grain-free formulation with a mix of novel proteins like duck and venison, combined with antioxidant-rich fruits and vegetables. This formulation is highly nutritious and supports overall wellness, but it is ranked third because it is priced at a premium level, making it less budget-friendly.

In addition, fourth formulation, “Budget-Friendly Grain-Free Blend”, offers an affordable alternative with plant-based protein sources such as pea protein and chickpeas. While it meets the grain-free requirement and remains cost-effective, it ranks lower due to the absence of joint-supporting ingredients, which were a priority for the pet owner.

Finally, fifth-ranked formulation, “Natural Raw-Infused Kibble”, features freeze-dried raw meat pieces blended with traditional kibble. Although this option provides superior protein quality and digestibility, it is ranked lowest due to its higher cost and limited availability in sustainable sourcing. By generating this ranked list, the ML model 108 helps the user 102 make an informed decision, balancing nutritional quality, cost considerations, and sustainability preferences.

Once the food formulations are ranked, the processing unit 210 generates the detailed report, using the ML model 108, for each of the plurality of food formulations. The report depicts at least one of: nutrient profile, ingredients list, cost analysis and sustainability metrics. The nutrient profile outlines macronutrient and micronutrient content, while the ingredient list ensures transparency about what goes into the animal's and pet's food. Additionally, the cost analysis helps users compare prices per unit, and the sustainability metrics assess factors like ingredient sourcing and carbon footprint.

The detailed report includes comparison of the generated plurality of food formulations against the plurality of pre-defined regulatory guidelines. For instance, if a generated food formulation is designed for adult dogs, the ML model 108 compares its nutrient profile—such as protein, fat, vitamins, and minerals—against the minimum and maximum thresholds set by AAFCO. If a formulation lacks the required level of calcium and phosphorus for bone health, the detailed report highlights this deficiency and suggests necessary adjustments. Similarly, if a formulation includes certain preservatives or additives restricted by FEDIAF, the ML model 108 flags this non-compliance and recommends alternative ingredients.

Additionally, the detailed report provides evaluation of each formulation's ingredient composition concerning permissible inclusion levels of specific nutrients. In addition, the detailed report includes list of one or more functional claims and one or more packaging-ready statements derived from one or more nutritional and functional attributes of the plurality of food formulations. The one or more functional claims may be “supports joint health”, “helps maintain healthy cartilage and joints”, and the lie. In an embodiment, the one or more functional claims are generated based on the functional attributes such as weight management, cost effectiveness, and the like.

Further, the one or more packaging-ready statements may be “made with 100% natural ingredients”, “rich in fish-oil”, “plant-based formulation”, “insect-based formulation”, etc. The one or more packaging-ready statements are generated based on the nutritional attributes such as glucosamine for joints or DHA for brain function, and the like. Also, the detailed report includes sustainability metrics and potential certifications including at least carbon footprint, water efficiency, and environmental impact metrics.

Further, the processing unit 210 is configured to generate one or more recommendations for reformulating one or more existing food formulations using the ML model 108. The ML model 108 facilitates generation of the one or more recommendations based on at least scientific research updates, regulatory updates, change in existing user preferences, and market pricing trends.

The scientific research updates include new studies that may reveal improved nutrient formulations, enhanced ingredient bioavailability, or additional health benefits of certain compounds. For instance, if recent scientific research demonstrates that a specific probiotic strain significantly enhances gut health in dogs, the ML model 108 analyses this and recommends incorporating the probiotic into existing formulations for digestive support. Further, the regulatory updates also play an important role in reformulation. Organizations such as AAFCO (Association of American Feed Control Officials) and FEDIAF (European Pet Food Industry Federation) frequently revise their guidelines, introducing new nutrient requirements or restricting certain ingredients. If AAFCO updates its standards to mandate higher Omega-3 levels in puppy food, the ML model 108 promptly identifies this change and recommends increasing DHA levels by incorporating fish oil or algae-based sources into the affected formulations. Further, if there is change in existing user preferences, for example, increasing demand for plant-based food, instead of animal-driven pet food, the ML model 108 recommends replacing animal-derived proteins, such as chicken meal, with plant-based alternatives.

Furthermore, the ML model 108 continuously monitors real-time market pricing trends, helping manufacturers make cost-effective ingredient substitutions. If the price of chicken meal surges by 20%, the ML model 108 analyzes alternative protein sources, such as insect protein or soy meal, and recommends suitable replacements that maintain nutritional balance while reducing costs.

In an example, let's assume that in 2023, a pet food manufacturer formulated an adult dog kibble recipe using chicken meal (28%) as a primary protein source, chicken fat (12%) for essential fatty acids, and brown rice and sweet potatoes as carbohydrate sources, with glucosamine added for joint health. However, in 2024, significant market and regulatory changes prompted the need for reformulation. Chicken meal prices surged by 30%, making the original formulation less cost-effective. Additionally, new research highlighted the benefits of turmeric extract for joint health, and AAFCO updated its guidelines to require higher Omega-3 levels in pet food. Using ML model 108, the processing unit 210 recommends replacing 10% of the chicken meal with pea protein to reduce costs while maintaining protein quality.

In addition, to meet the new Omega-3 requirements, the ML model 108 recommends incorporating flaxseed oil and introducing turmeric extract to enhance joint health benefits, complementing the existing glucosamine supplement. This reformulation not only ensures compliance with updated industry regulations but also reduced raw material costs by 12%, making the pet food formulation more competitive while improving health benefits and maintaining consumer trust. In an embodiment, the ML model 108 may be a part of the processing unit 210 of the system 106. In another embodiment, the ML model 108 is integrated in a separate entity present outside the system 106 but associated with the system 106 through the communication network 110.

To generate the one or more recommendations for reformulating the one or more existing food formulations, the ML model 108 follows a structured process. First, the ML model 108 collects and monitors real-time data from regulatory databases, scientific research publications, ingredient suppliers, and market analytics. The ML model 108 evaluates impact of these changes on key parameters such as nutritional adequacy, regulatory compliance, palatability, and cost. Using advanced optimization techniques, the ML model 108 recommends adjusting the existing food formulations by rebalancing ingredients and nutrient levels while preserving product integrity. For example, if new research highlights the benefits of turmeric extract for joint health, the ML model 108 suggests adding it to senior dog formulations to enhance mobility support.

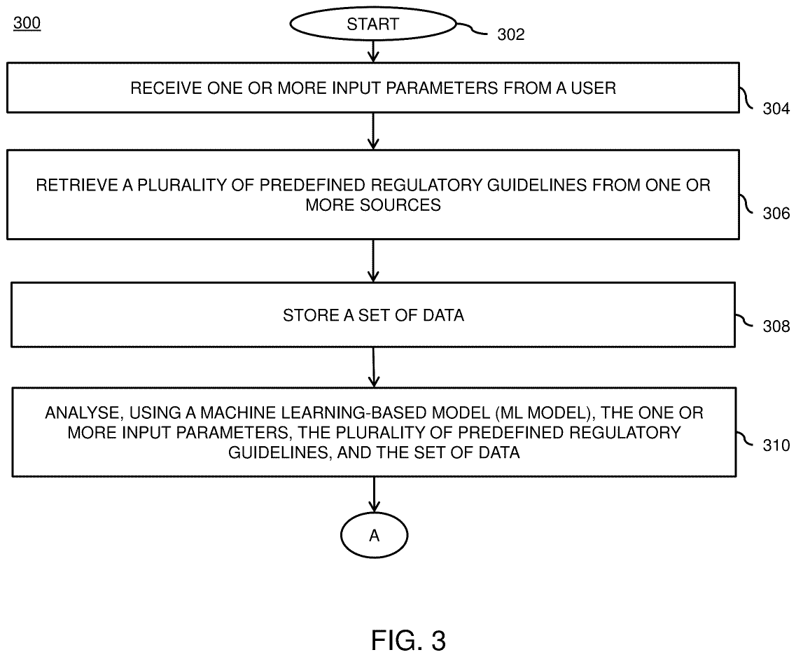

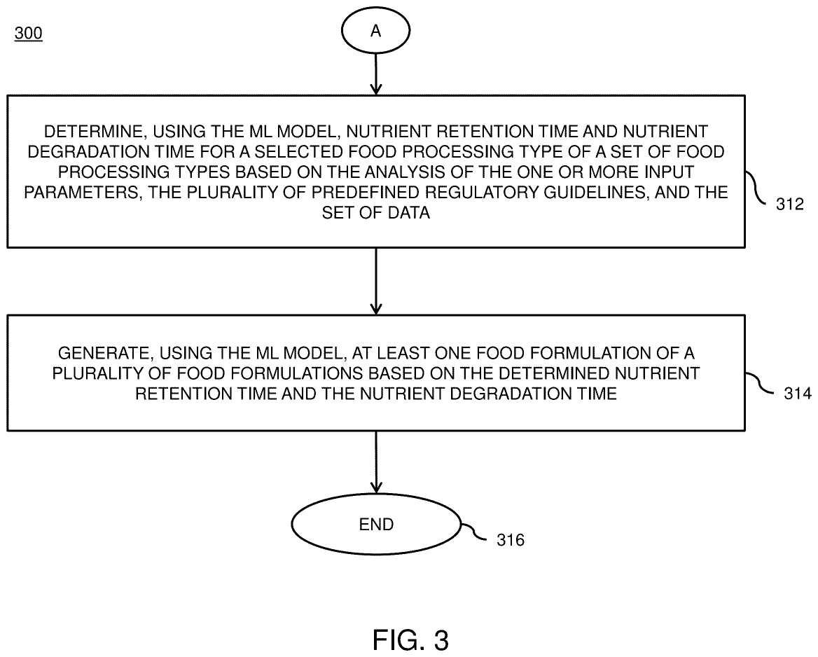

FIG. 3 illustrates a flowchart 300 of a method for generating the food formulations, in accordance with various embodiments of the present disclosure. It may be noted that the description of the flowchart 300 refers to FIG. 1 and FIG. 2. The working and functioning may be read from the description of FIG. 1 and FIG. 2.

The flowchart 300 initiates at step 302. At step 304, the method includes receiving the one or more input parameters from the user 102. The one or more input parameters include animal and pet category, animal and pet food habits, intended nutritional goals, geographical location, ingredient preferences, ingredient exclusions, and the set of food processing types.

At step 306, the method includes retrieving the plurality of pre-defined regulatory guidelines based on the one or more user-defined parameters from the one or more data sources 112. The one or more sources includes at least one of: regulatory databases, ingredient databases, cost datasets, environmental impact datasets, and animal and pet behaviour studies.

At step 308, the method includes storing the set of data. The set of data includes at least a plurality of nutritional profiles, environmental impact metrics, cost-related data, palatability metrics, and historical nutrient retention coefficients.

At step 310, the method includes analysing the one or more input parameters, the plurality of pre-defined pet nutrition-based regulatory guidelines, and the set of data using the machine learning-based model 108 (ML model 108).

At step 312, the method includes determining the nutrient retention time and the nutrient degradation time for the selected food processing type of the set of food processing types using the ML model 108. The nutrient retention time and the nutrient degradation time is determined based on the analysis of the one or more input parameters, the plurality of pre-defined pet nutrition-based regulatory guidelines, and the set of data.

At step 314, the method includes generating, using the ML model 108, at least one pet food formulation of the plurality of food formulations based on the determined nutrient retention time and the nutrient degradation time. The plurality of food formulations includes one of: pet food formulations and animal feed formulations.

The flowchart 300 ends at step 316. It may be noted that the flowchart 300 includes the above stated process steps. However, there may be more number of steps in the flowchart 300.

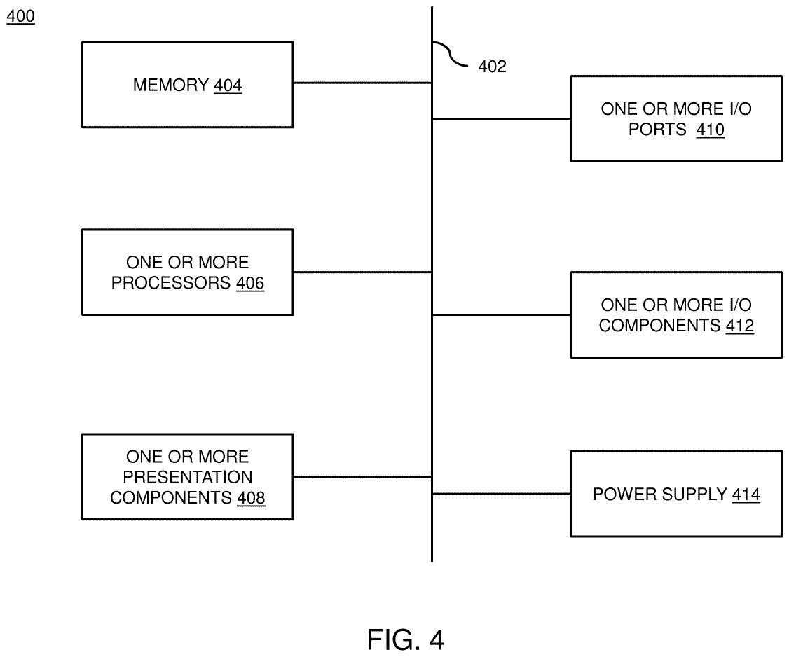

FIG. 4 illustrates an exemplary block diagram of a computing device 400, in accordance with various embodiments of the present disclosure. The block diagram illustrates a general architecture for the computing device 400 that may be employed to implement various elements of the systems and methods described and illustrated herein. The computing device 400 can be used to implement the system 106, the user device 104, the ML model 108, the database 208, etc. The computing device 400 is a non-transitory computer-readable storage medium. For example, the non-transitory computer-readable storage medium can include media such as magnetic media (such as hard disks, floppy disks, etc.), optical media (such as compact discs, digital video discs, Blu-ray discs, etc.), semiconductor media (such as flash memory, electrically programmable read only memory (EPROM), electrically erasable programmable read only memory (EEPROM), etc.), any suitable media that is not fleeting or devoid of any semblance of permanence during transmission, and/or any suitable tangible media. In an implementation, the computing device 400 is identical to the system 106 of FIG. 1. The computing device 400 includes a bus 402 that directly or indirectly couples the following devices: memory 404, one or more processors 406, one or more presentation components 408, one or more input/output (I/O) ports 410, one or more input/output components 412, and an illustrative power supply 414. The bus 402 represents what may be one or more buses (such as an address bus, data bus, or combination thereof). Although the various blocks of FIG. 4 are shown with lines for the sake of clarity, in reality, delineating various components is not so clear, and metaphorically, the lines would more accurately be grey and fuzzy. For example, one may consider a presentation component such as a display device to be an I/O component. Also, processors have memory. The inventors recognize that such is the nature of the art and reiterate that the diagram of FIG. 4 is merely illustrative of the exemplary computing device 400 that can be used in connection with one or more embodiments of the present disclosure. Distinction is not made between such categories as “workstation,” “server,” “laptop,” “hand-held device,” etc., as all are contemplated within the scope of FIG. 4 and reference to “device.”

The computing device 400 typically includes a variety of computer-readable media. The computer-readable media can be any available media that can be accessed by the computing device 400 and includes both volatile and nonvolatile media, removable and non-removable media. By way of example, and not limitation, the computer-readable media may comprise computer storage media and communication media. The computer storage media includes volatile and nonvolatile, removable and non-removable media implemented in any method or technology for storage of information such as computer-readable instructions, data structures, program modules or other data. The computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by the computing device 400. The communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer-readable media.

The memory 404 includes computer-storage media in the form of volatile and/or non-volatile memory. The memory 404 may be removable, non-removable, or a combination thereof. Exemplary hardware devices include solid-state memory, hard drives, optical-disc drives, etc. The computing device 400 includes the one or more processors 406 that read data from various entities such as the memory 404 or the I/O components 412. The one or more presentation components 408 present data indications to a user or other device. Exemplary presentation components include a display device, speaker, printing component, vibrating component, etc. The one or more I/O ports 410 allow the computing device 400 to be logically coupled to other devices including the one or more I/O components 412, some of which may be built in. Illustrative components include a microphone, joystick, game pad, satellite dish, scanner, printer, wireless device, etc.

The computing device 400 can be of varying types including a workstation, server (the system 106), computing cluster, blade server, server farm, or any other data processing system or computing device. Due to the ever-changing nature of computers and networks, the description of the computing device 400 depicted in FIG. 4 is intended only as a specific example for purposes of illustrating some implementations. Many other configurations of the computing device 400 are possible having more or fewer components than the computing device depicted in FIG. 4.

In an aspect, the present disclosure relates to a non-transitory computer-readable medium storing one or more instructions for generating the pet food formulations. The one or more instructions which, when executed by a processor (similar to the processing unit 210), causes the processor to receive the one or more input parameters from the user 102. The one or more instructions which, when executed, causes the processor to retrieve the plurality of pre-defined regulatory guidelines from the one or more sources 112. In addition, the one or more instructions which, when executed, causes the processor to store a set of data, wherein the set of data comprises at least a plurality of nutritional profiles, environmental impact metrics, cost-related data, and historical nutrient retention coefficients. Further, the one or more instructions which, when executed, causes the processor to analyse, using the machine learning-based model 108, the one or more input parameters, the plurality of pre-defined pet nutrition-based regulatory guidelines, and the set of data. Also, the one or more instructions which, when executed, causes the processor to determine, using the machine learning-based model 108, the nutrient retention time and nutrient degradation time for a selected food processing type of a set of food processing types. The nutrient retention time and the nutrient degradation time is determined based on the analysis of the one or more input parameters, the plurality of pre-defined pet nutrition-based regulatory guidelines, and the set of data. The one or more instructions which, when executed, causes the processor to generate, using the machine learning-based model 108, at least one food formulation of the plurality of food formulations based on the determined nutrient retention time and the nutrient degradation time.

The present disclosure provides various advantages. The present disclosure provides a highly efficient and intelligent approach to generate food formulations, leveraging machine learning (ML) model to ensure optimal nutrition, regulatory compliance, and cost-effectiveness. One of the key advantages is its ability generate food formulations based on user preferences, and regulatory guidelines. Another key advantage of the present disclosure is to dynamically reformulate formulations based on real-time data, including scientific research updates, regulatory updates, and ingredient availability. This ensures that animal and pet food products remain up-to-date with the latest industry standards while maintaining high nutritional quality. Additionally, the system's ability to rank multiple formulations based on user preferences allows manufacturers, veterinarians, and pet owners to make well-informed decisions tailored to specific dietary needs, such as hypoallergenic diets, weight management, or medical conditions.

Another major advantage of the disclosure is to improve efficiency and reduce pet food formulation costs. By continuously analyzing ingredient pricing trends and sustainability metrics, the system suggests cost-effective yet nutritionally balanced alternatives, helping manufacturers maintain profitability without compromising quality. Moreover, the automated compliance check against regulatory guidelines reduces the risk of non-compliance, ensuring smoother market approvals and reducing the need for costly reformulations. The invention also enhances customization, allowing pet owners or nutritionists to create personalized diets that cater to individual animals and pets based on factors such as breed, age, location, and dietary preferences, ultimately improving pet health and well-being.

The foregoing descriptions of specific embodiments of the present technology have been presented for the purposes of illustration and description. They are not intended to be exhaustive or to limit the present technology to the precise forms disclosed, and obviously many modifications and variations are possible in light of the above teaching. The embodiments were chosen and described in order to best explain the principles of the present technology and its practical application, to thereby enable others skilled in the art to best utilize the present technology and various embodiments with various modifications as are suited to the particular use contemplated. It is understood that various omissions and substitutions of equivalents are contemplated as circumstance may suggest or render expedient, but such are intended to cover the application or implementation without departing from the spirit or scope of the claims of the present technology.

While several possible embodiments of the disclosure have been described above and illustrated in some cases, it should be interpreted and understood as to have been presented only by way of illustration and example, but not by limitation. Thus, the breadth and scope of a preferred embodiment should not be limited by any of the above-described exemplary embodiments.

What is claimed is:

1. A system for generating food formulations, the system comprising:
an input module configured to receive one or more input parameters from a user;
a regulatory data integration module configured to retrieve a plurality of pre-defined regulatory guidelines from one or more sources;
a database configured to store a set of data, wherein the set of data comprises at least a plurality of nutritional profiles, environmental impact metrics, cost-related data, palatability metrics, and historical nutrient retention coefficients; and
a processing unit configured to:
analyse, using a machine learning-based model, the one or more input parameters, the plurality of pre-defined regulatory guidelines, and the set of data;
determine, using the machine learning-based model, nutrient retention time and nutrient degradation time for a selected food processing type of a set of food processing types based on the analysis of the one or more input parameters, the plurality of pre-defined nutrition-based regulatory guidelines, and the set of data; and
generate, using the machine learning-based model, at least one food formulation of a plurality of food formulations based on the determined nutrient retention time and the nutrient degradation time.

2. The system of claim 1, wherein the plurality of food formulations comprises one of: pet food formulations and animal feed formulations.

3. The system of claim 1, wherein the one or more input parameters comprises animal and pet category, animal and pet food habits, intended nutritional goals, geographical location, ingredient preferences, ingredient exclusions, and the set of food processing types.

4. The system of claim 1, wherein the set of data is extracted from the one or more sources, wherein the one or more sources comprises at least one of: regulatory databases, ingredient databases, cost datasets, environmental impact datasets, and animal and pet behaviour studies.

5. The system of claim 1, wherein the processing unit is configured to rank, using the machine learning-based model, the plurality of food formulations based on a set of user preferences, wherein the set of user preferences is determined based on the one or more input parameters received from the user.

6. The system of claim 1, wherein the processing unit is configured to generate, using the machine learning-based model, a detailed report for each of the plurality of food formulations depicting at least one of: nutrient profile, ingredients list, cost and sustainability metrics.

7. The system of claim 6, wherein the detailed report comprises:
comparison of the generated plurality of food formulations against the plurality of pre-defined regulatory guidelines,
a list of one or more functional claims and one or more packaging-ready statements derived from one or more nutritional and functional attributes of the plurality of food formulations, and
sustainability metrics and potential certifications comprising at least carbon footprint, water efficiency, and environmental impact metrics.

8. The system of claim 1, wherein the machine learning-based model is trained using pre-historic data and real-time data associated with the one or more input parameters, wherein the pre-historic data is stored in the database and the real time data is received from at least one of: the one or more sources and the user.

9. The system of claim 1, wherein the processing unit is further configured to generate, using the machine learning-based model, one or more recommendations for reformulating one or more existing food formulations, based on at least a scientific research, regulatory updates, change in existing user preferences, and market pricing trends.

10. The system of claim 1, wherein the database is periodically updated based on at least a scientific research, regulatory updates, change in existing user preferences, and market pricing trends.

11. A method for generating food formulations, comprising:
receiving one or more input parameters from a user;
retrieving a plurality of pre-defined regulatory guidelines from one or more sources;
storing a set of data comprising at least a plurality of nutritional profiles, environmental impact metrics, cost-related data, palatability metrics, and historical nutrient retention coefficients;
analysing, using a machine learning-based model, the one or more input parameters, the plurality of pre-defined nutrition-based regulatory guidelines, and the set of data;
determining, using the machine learning-based model, nutrient retention time and nutrient degradation time for a selected food processing type of a set of food processing types based on the analysis of the one or more input parameters, the plurality of pre-defined nutrition-based regulatory guidelines, and the set of data; and
generating, using a machine learning-based model, at least one food formulation of a plurality of food formulations based on the determined nutrient retention time and the nutrient degradation time.

12. The method of claim 11, wherein the plurality of food formulations comprises one of: pet food formulations and animal feed formulations.

13. The method of claim 11, wherein the one or more input parameters comprises animal and pet category, animal and pet food habits, intended nutritional goals, geographical location, ingredient preferences, ingredient exclusions, and the set of food processing types.

14. The method of claim 11, further comprising extracting the set of data from the one or more sources, wherein the one or more sources comprises at least one of: regulatory databases, ingredient databases, cost datasets, environmental impact datasets, and animal and pet behaviour studies.

15. The method of claim 11, further comprising ranking, via the machine learning-based model, the plurality of food formulations based on a set of user preferences, wherein the set of user preferences is determined based on the one or more input parameters received from the user.

16. The method of claim 11, further comprising: generating, via the machine learning-based model, a detailed report for each of the plurality of food formulations depicting at least one of: nutrient profile, ingredients list, cost and sustainability metrics.

17. The method of claim 16, wherein the detailed report comprises:
comparison of the generated plurality of food formulations against the plurality of pre-defined nutrition-based regulatory guidelines,
list of one or more functional claims and one or more packaging-ready statements derived from one or more nutritional and functional attributes of the plurality of food formulations, and
sustainability metrics and potential certifications comprising at least carbon footprint, water efficiency, and environmental impact metrics.

18. The method of claim 11, further comprising training the machine learning-based model using pre-historic data and real-time data associated with the one or more input parameters, wherein the pre-historic data is stored in a database and the real time data is received from at least one of: the one or more sources and the user.

19. The method of claim 11, further comprising: generating, using the machine learning-based model, one or more recommendations for reformulating one or more existing food formulations based on at least a scientific research, regulatory updates, change in existing user preferences, and market pricing trends.

20. A non-transitory computer-readable medium storing one or more instructions for generating food formulations, wherein the one or more instructions which, when executed by a processor, causes the processor to:
receive one or more input parameters from a user;
retrieve a plurality of pre-defined regulatory guidelines from one or more sources;
store a set of data, wherein the set of data comprises at least a plurality of nutritional profiles, environmental impact metrics, cost-related data, and historical nutrient retention coefficients;
analyse, using a machine learning-based model, the one or more input parameters, the plurality of pre-defined nutrition-based regulatory guidelines, and the set of data;
determine, using the machine learning-based model, nutrient retention time and nutrient degradation time for a selected food processing type of a set of food processing types based on the analysis of the one or more input parameters, the plurality of pre-defined nutrition-based regulatory guidelines, and the set of data; and
generate, using a machine learning-based model, at least one food formulation of a plurality of food formulations based on the determined nutrient retention time and the nutrient degradation time.
